(19) United States
(12) Patent Application Publication
(10) Pub. No.: US 2024/0191418 A1
(43) Pub. Date: Jun. 13, 2024

(54) SANITIZING ASSEMBLY FOR A CLEANING APPLIANCE HAVING AN INTERNAL PROCESSING SPACE FOR SANITIZING ARTICLES

(71) Applicant: WHIRLPOOL CORPORATION, Benton Harbor, MI (US)

(72) Inventors: Raosaheb Ananda Farakte, Pune (IN); Amol Sopan Kolambe, Pune (IN); Ajay Manik Naiknaware, Pune (IN); Rajwardhan Ajit Deshmukh, Sangli (IN); Swapnil Jani, Ahmedabad (IN); Nitin Minocha, Pune (IN)

(73) Assignee: WHIRLPOOL CORPORATION, BENTON HARBOR, MI (US)

(21) Appl. No.: 18/076,668

(22) Filed: Dec. 7, 2022

(51) Int. Cl. D06F 35/00; A61L 2/025; A61L 2/26; B06B 1/06; B08B 3/04; D06F 23/04; D06F 37/12

(52) U.S. Cl. CPC D06F 35/002; A61L 2/025; A61L 2/26; B06B 1/0637; B08B 3/045; D06F 23/04; D06F 37/12; A61L 2202/11; A61L 2202/26; B06B 2201/71; B08B 2203/007

(57) ABSTRACT

A laundry appliance includes a drum that rotationally operates within a tub. A fluid delivery system selectively delivers wash fluid to the drum. A rotator rotationally operates within the drum. The rotator selectively operates independent of the drum and about a common rotational axis of the drum and the rotator. A sanitizing mechanism includes an ultrasonic transducer. The ultrasonic transducer selectively delivers waves of an ultrasonic frequency into an amount of the wash fluid disposed within the drum and the rotator. The ultrasonic frequency generates air bubbles and causes cavitation of the air bubbles that directs a micro jet of air through the wash fluid and into articles being processed within the drum.

### BACKGROUND OF THE DISCLOSURE

The present disclosure generally relates to the field of cleaning appliances, such as a laundry appliance and other appliances that include a processing space within which articles can be cleaned and sanitized. More specifically, the device is in the field of a sanitizing mechanism that incorporates one or more ultrasonic transducers that operate to produce microbubbles within a wash fluid, wherein the microbubbles can be cavitated to produce micro jets of air for cleaning and sanitizing articles within a processing space.

### SUMMARY OF THE DISCLOSURE

According to one aspect of the present disclosure, a laundry appliance includes a drum that rotationally operates within a tub. A fluid delivery system selectively delivers wash fluid to the drum. A rotator rotationally operates within the drum. The rotator selectively operates independent of the drum and about a common rotational axis of the drum and the rotator. A sanitizing mechanism includes an ultrasonic transducer. The ultrasonic transducer selectively delivers waves of an ultrasonic frequency into an amount of the wash fluid disposed within the drum and the rotator. The ultrasonic frequency generates air bubbles and causes cavitation of the air bubbles that directs a micro jet of air through the wash fluid and into articles being processed within the drum.

According to another aspect of the present disclosure, an agitator for a laundry appliance includes a lower section that includes raised fins, a post that extends upward from the lower section, and an ultrasonic transducer assembly that is attached to the post. The ultrasonic transducer assembly is positioned to direct waves of an ultrasonic frequency in a three-dimensional configuration that radiates from the post. The ultrasonic transducer assembly is selectively removable from a receiver that is defined within the post. The ultrasonic transducer assembly delivers the waves of the ultrasonic frequency into an amount of wash fluid that is disposed within a processing space. The waves of the ultrasonic frequency are configured to generate air bubbles and cause cavitation of the air bubbles that directs a micro jet of air through the wash fluid and into articles that are processed within the processing space.

According to yet another aspect of the present disclosure, a cleaning appliance includes a tub disposed within a structural cabinet. The tub defines a processing space. A fluid delivery system selectively delivers wash fluid to the processing space. A rotator rotationally operates within the processing space. The rotator selectively rotates within the processing space about a rotational axis. A sanitizing mechanism includes an ultrasonic transducer. The ultrasonic transducer selectively delivers waves of an ultrasonic frequency into an amount of the wash fluid disposed within the processing space. The waves of the ultrasonic frequency generate air bubbles and cause cavitation of the air bubbles that directs a micro jet of air through the wash fluid and into articles that are processed within the processing space. The sanitizing mechanism is selectively removable from a receiver that is defined within the rotator.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is a top perspective view of a laundry appliance that incorporates an aspect of a sanitizing mechanism;

FIG. 2 is a partial cross-sectional view of a vertical axis laundry appliance showing an exemplary orientation of the sanitizing mechanism with respect to the processing space and the wash fluid within the processing space;

FIG. 3 is an enlarged perspective view of an aspect of the sanitizing mechanism for producing the waves of the ultrasonic frequency within a wash fluid;

FIG. 4 is a schematic diagram illustrating operation of the sanitizing mechanism to produce microbubbles and cavitate these microbubbles for generating the micro jets of air within the wash fluid;

FIG. 5 is a schematic diagram illustrating operation of the micro jets with respect to an article being processed through use of the sanitizing mechanism;

FIG. 6 is a schematic diagram illustrating an exemplary wash cycle that incorporates an aspect of a sanitizing phase for sanitizing articles during a wash cycle within an appliance;

FIG. 7 is a schematic diagram illustrating operation of a particular sanitizing phase or a dedicated sanitizing cycle for an appliance;

FIG. 8 is a schematic diagram illustrating an appliance that incorporates an aspect of the sanitizing mechanism within an agitator of the appliance;

FIG. 9 is a top perspective view of an appliance that incorporate an aspect of the sanitizing mechanism within an operable lid of the appliance;

FIG. 10 is a perspective view of a front-load appliance that incorporates an aspect of the sanitizing mechanism within a door of the appliance; and

FIG. 11 is a linear flow diagram illustrating a method for operating a sanitizing mechanism within a cleaning appliance.

The components in the figures are not necessarily to scale, emphasis instead being placed upon illustrating the principles described herein.

### DETAILED DESCRIPTION

The present illustrated embodiments reside primarily in combinations of method steps and apparatus components related to a cleaning appliance having a sanitizing mechanism incorporated within a processing space of the appliance for producing microbubbles and also cavitating these microbubbles to produce micro jets of air that can be used for sanitizing articles to be processed within the processing space. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

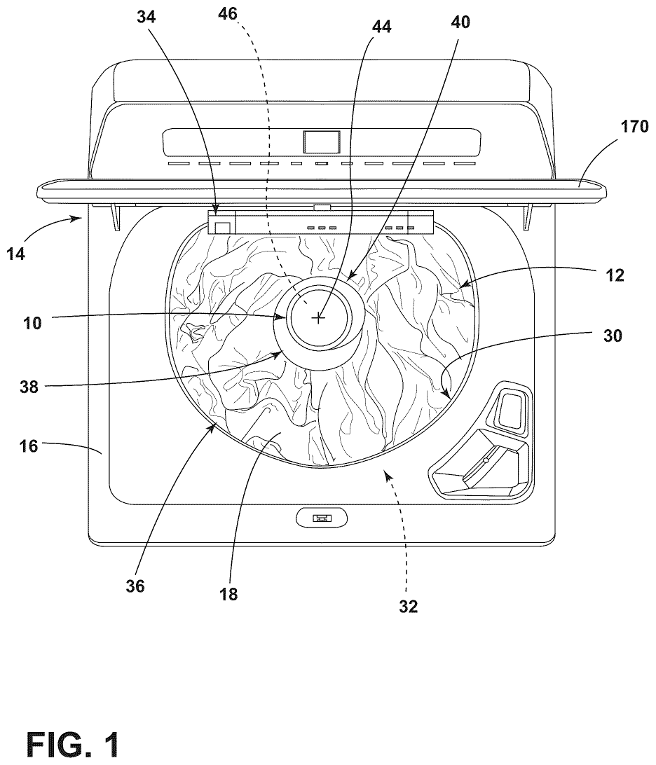

For purposes of description herein, the terms “upper,” “lower,” “right,” “left,” “rear,” “front,” “vertical,” “horizontal,” and derivatives thereof shall relate to the disclosure as oriented in FIG. 1. Unless stated otherwise, the term “front” shall refer to the surface of the element closer to an intended viewer, and the term “rear” shall refer to the surface of the element further from the intended viewer. However, it is to be understood that the disclosure may assume various alternative orientations, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

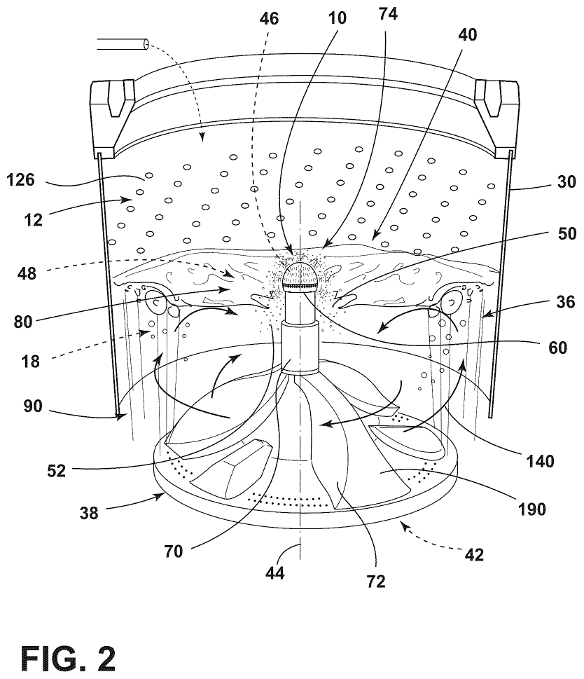

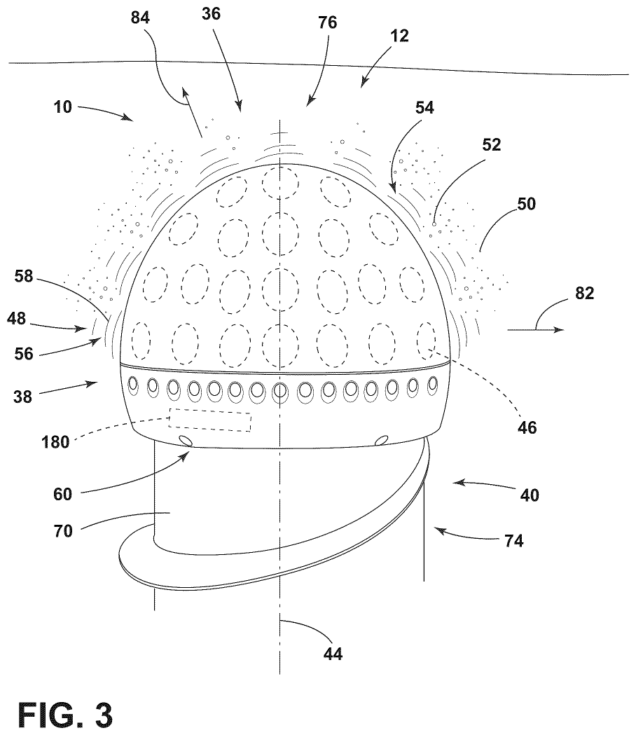

Referring to FIGS. 1-7, reference numeral 10 generally designates a sanitizing mechanism that is incorporated within a processing space 12 of an appliance 14. The appliance 14 can be in the form of a cleaning appliance having the processing space 12 positioned within an outer cabinet 16. This processing space 12 is used for cleaning and sanitizing various articles 18 within the appliance 14. These appliances 14 can include, but are not limited to, laundry washing appliances, combination laundry washing and drying appliances, dishwashers, refreshing and sanitizing appliances, and other similar cleaning appliances that include a processing space 12 within which articles 18 can be processed.

Referring again to FIGS. 1-7, with reference to a laundry appliance 14, the laundry appliance 14 can include a drum 30 that rotationally operates within a tub 32. A fluid delivery system 34 is incorporated within the appliance 14 for selectively delivering wash fluid 36 into the processing space 12 that is at least partially defined by the drum 30. A rotator 38, typically in the form of an agitator 40 or impeller 42 is disposed within the drum 30. The rotator 38 rotationally operates within the drum 30. Additionally, the rotator 38 is configured to selectively operate independent of the drum 30 about a common rotational axis 44 of both the drum 30 and the rotator 38. In this manner, the rotator 38 and the drum 30 can selectively operate in unison. The rotator 38 and the drum 30 can also selectively operate in opposite directions, at different speeds, according to different operational patterns and configurations, combinations thereof, and other coordinating rotational movements about the common rotational axis 44. The sanitizing mechanism 10 is typically attached to the rotator 38 and includes an ultrasonic transducer 46.

Referring again to FIGS. 1-7, the ultrasonic transducer 46 selectively delivers waves 48 of an ultrasonic frequency into an amount of wash fluid 36 that is disposed within the drum 30. The wash fluid 36 within the drum 30 surrounds the rotator 38 and, in certain aspects of the device, can also enter and pass through portions of the rotator 38. The waves 48 of the ultrasonic frequency are used to generate air bubbles 50, typically small microbubbles 52, within the wash fluid 36. The waves 48 of the ultrasonic frequency also cause cavitation 54 of the air bubbles 50 that collapse the air bubbles 50 and produce micro jets 56 of air 58 that are directed through the wash fluid 36 and toward and/or into the articles 18 being processed within the processing space 12 defined by the drum 30. It is contemplated that the sanitizing mechanism 10 that includes the ultrasonic transducer 46 is selectively removable from the rotator 38. In this manner, the rotator 38 can include a receiver 60 that is used to attach the sanitizing mechanism 10 having the one or more ultrasonic transducers 46 to the rotator 38.

Referring again to FIGS. 1-7, the sanitizing mechanism 10 can be attached to an agitator 40 that includes a post 70 that extends upward from an impeller 42 portion of the agitator 40. In certain aspects of the device, the post 70 can be a member that is attached to a base 72 of the agitator 40, where the base 72 resembles an impeller 42. In certain aspects of the device, the post 70 can be removed from the base 72 of the rotator 38 such that the rotator 38 can be alternated between an impeller 42 configuration and an agitator 40 configuration. Where the post 70 is used as part of the rotator 38, the sanitizing mechanism 10 can be incorporated within the post 70 or other cylindrical portion of the agitator 40. The sanitizing mechanism 10 can also be disposed at a top portion 74 of the agitator 40, such as the top of the post 70 for the agitator 40.

In the various configurations of the rotator 38, as described herein, the location of the sanitizing mechanism 10 and the ultrasonic transducers 46 are located on the rotator 38 such that the ultrasonic transducers 46 are submerged, or at least partially submerged beneath the surface of the wash fluid 36. Typically, the ultrasonic transducers 46 are positioned on the rotator 38 to be sufficiently beneath the surface of the wash fluid 36 to produce the three dimensional configuration 80 of the waves 48 that radiate outward from the sanitizing mechanism 10 for acting on the wash fluid 36 and the articles 18 being processed. In this manner, the ultrasonic transducers 46 can act upon saturated or submerged articles 18 that may be above, below or adjacent to the sanitizing mechanism 10.

In each of these configurations, the sanitizing mechanism 10 can be in the form of a generally spheroid member 76 having a curved outer surface 78. The ultrasonic transducers 46 of the sanitizing mechanism 10 are oriented along this curved outer surface 78. In this manner, the waves 48 of the ultrasonic frequency can be produced within a portion of the sanitizing mechanism 10 and can radiate outward in a three dimensional configuration 80 from the outer surface 78 of the sanitizing mechanism 10 and into the wash fluid 36 within the processing space 12. In this manner, the generally spheroid configuration of the sanitizing mechanism 10 and the ultrasonic transducers 46 produce waves 48 of the ultrasonic frequency that radiate in the three-dimensional configuration 80 that includes lateral directions 82 and vertical directions 84 and combinations of lateral and vertical directions 82, 84. This three-dimensional configuration 80 can be a generally semi-spherical configuration, a generally spherical configuration, or other similar three-dimensional configurations 80 that can radiate the waves 48 of ultrasonic frequency throughout the wash fluid 36.

By way of example, and not limitation, the generally spheroid configuration of the sanitizing mechanism 10 can produce the waves 48 of the ultrasonic frequency that can emanate in a 360° path around the sanitizing mechanism 10. Additionally, depending upon the exact configuration of the sanitizing mechanism 10, these waves 48 of the ultrasonic frequency can also radiate upward and downward through the wash fluid 36 within the processing space 12. Through this configuration, the waves 48 of the ultrasonic frequency can radiate in all directions from the sanitizing mechanism 10 and through the wash fluid 36 for acting on the wash fluid 36 that is disposed within and around the articles 18 being processed. Again, the waves 48 of the ultrasonic frequency produce the air bubbles 50 and the cavitation 54 that, in turn, produces the micro jets 56 of air 58 that is directed at the articles 18 being processed.

In certain aspects of the device, the sanitizing mechanism 10 can be incorporated onto to a top portion 74 of the impeller 42 for the appliance 14. In such a configuration, the sanitizing mechanism 10 is positioned within a lower portion 90 of the processing space 12 and can be used to provide the waves 48 of ultrasonic frequency in a generally lateral direction 82 and vertical direction 84, and directions therebetween to produce the three-dimensional configuration 80 of the waves 48 for producing air bubbles 50 and the cavitation 54 that produces the micro jets 56 of air 58 within the wash fluid 36.

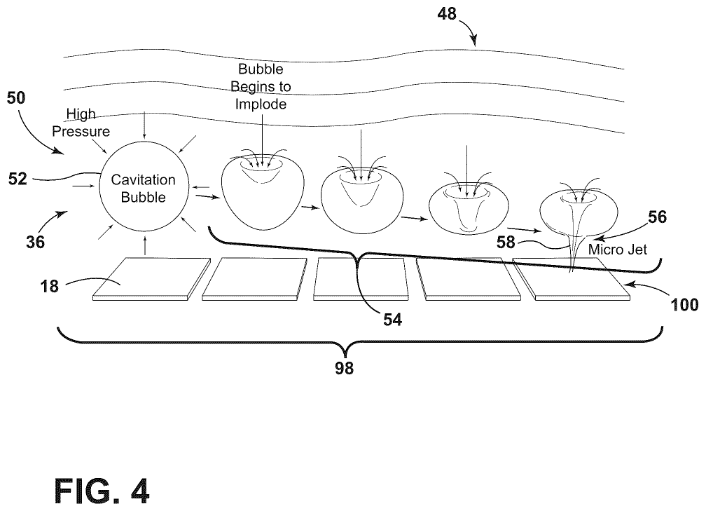

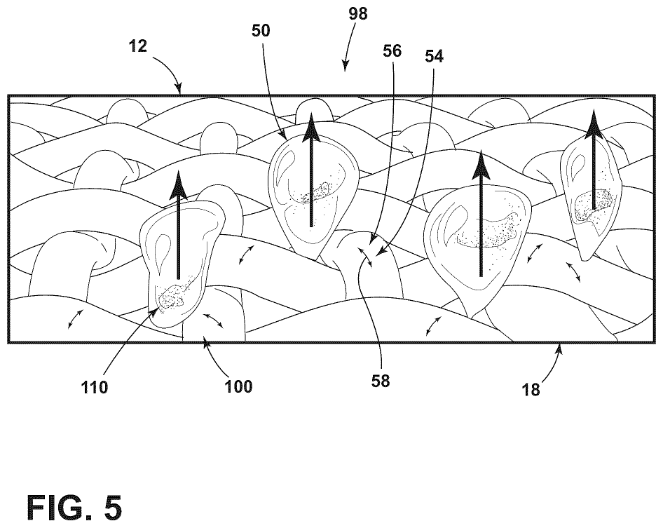

Referring now to FIGS. 4 and 5, during operation of the sanitizing mechanism 10, the ultrasonic transducers 46 produce the waves 48 of the ultrasonic frequency that are directed in a plurality of directions within the wash fluid 36. These waves 48 of the ultrasonic frequency produce the microbubbles 52 within the wash fluid 36 in areas near the sanitizing mechanism 10. As described herein, the use of the waves 48 of the ultrasonic frequency also cause a cavitation 54 action that implodes the various microbubbles 52, as shown in FIG. 4. This implosion, or cavitation 54, causes a collapse of the microbubble 52 and produces a micro jet 56 of air 58 that is directed from the now collapsed microbubble 52 and toward the articles 18 being processed.

During use of the sanitizing mechanism 10, the articles 18 being processed can slowly be rearranged through rotational operation of the rotator 38 and the drum 30. Through this rearrangement of the articles 18, different portions of the articles 18 are positioned adjacent to the sanitizing mechanism 10 through the sanitizing phase 98 of the appliance 14. During operation of the ultrasonic transducers 46 of the sanitizing mechanism 10, microbubbles 52 are produced and cavitation 54 occurs with respect to different treated portions 100 of the articles 18 throughout the course of the sanitizing phase 98. In this manner, throughout the sanitizing phase 98 of the appliance 14, most if not all of the various portions of the articles 18 being processed are acted on by the microbubbles 52 and the micro jets 56 of air 58 that are produced through the cavitation 54 of these microbubbles 52. Accordingly, at the conclusion of the sanitizing phase 98, the treated portions 100 of the articles 18 define most, if not all, of the surfaces of the articles 18.

Using these micro jets 56 of air 58, particles 110 of varying sizes can be dislodged, removed, or otherwise separated from the articles 18 being processed. According to various aspects of the device, the directions of the micro jets 56 of air 58 that result from the cavitation 54 of the microbubbles 52 can be generally random such that these micro jets 56 of air 58 are produced in all directions. Accordingly, within those treated portions 100 of the articles 18 being processed that are near the sanitizing mechanism 10, the various micro jets 56 of air 58 radiate in all directions within, around, and through the material of the articles 18 being processed.

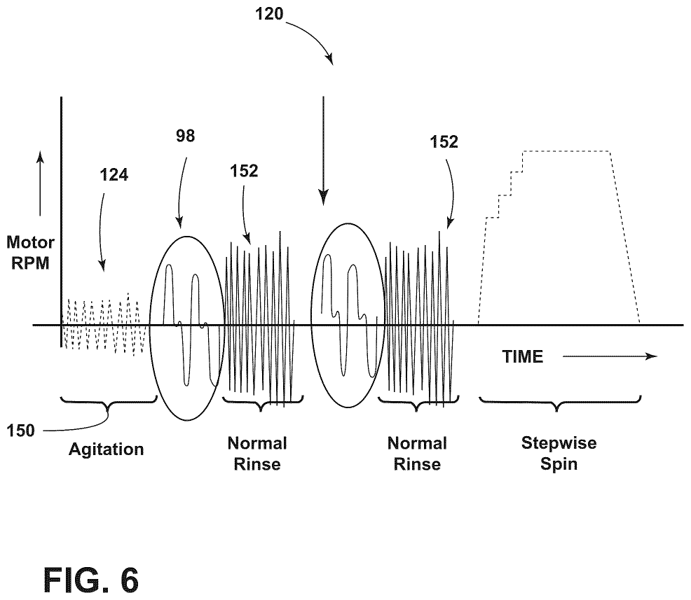

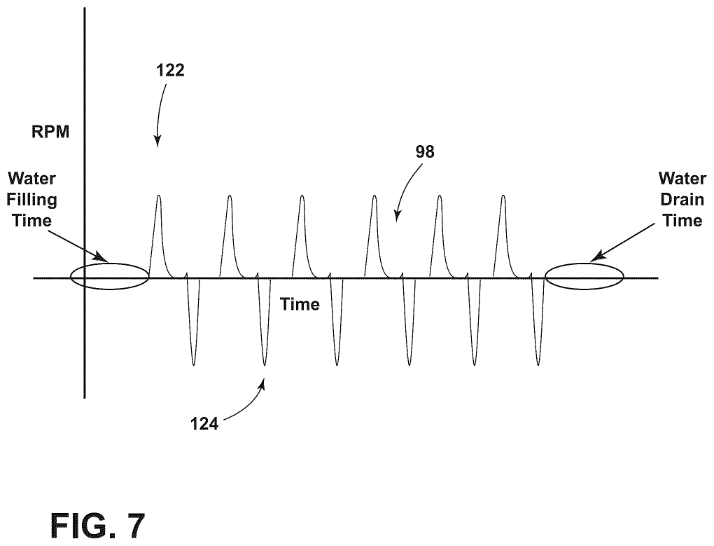

Referring now to FIGS. 6 and 7, the sanitizing phase 98 of the laundry appliance 14 can be a particular portion of a wash cycle 120, as shown in FIG. 6. The sanitizing phase 98 can also be a dedicated cycle 122 within the laundry appliance 14, as shown in FIG. 7. In each of these instances, the sanitizing phase 98 of the appliance 14 typically utilizes a continuous activation, or a series or pattern of activations, of the ultrasonic transducers 46 in combination with a rotation of the rotator 38 and, in certain instances, the drum 30. In configurations where the drum 30 is rotated, the agitator 40 is also rotated within the drum 30 to produce an agitating function 124 that rearranges the articles 18 within the processing space 12. The rotation of the drum 30 is typically operated through a slow rotation to minimize the effect of any centrifugal force that may be produced through the rotation of the drum 30. The sanitizing phase 98 is meant to direct the articles 18 toward the agitator 40. A rotation of the drum 30 above a particular rotational speed may produce a centrifugal force that causes the articles 18 to move away from the rotator 38 and toward a perforated wall 126 of the drum 30.

According to the various aspects of the device, the operation of the rotator 38 and the drum 30 about the common rotational axis 44 produces a toroidal flow 140 of the wash fluid 36 that assists in generating the agitating function 124 and rearrangement of the articles 18 being processed. In this manner, the articles 18 typically move in a similar toroidal flow 140 that progressively moves the articles 18 through the processing space 12 in a cyclical motion. This motion, in an exemplary aspect, operates the wash fluid 36 and the articles 18 along the path of the toroidal flow 140 and toward the agitator 40, then away from the agitator 40, and progressively back toward the agitator 40 to follow this toroidal flow 140 of wash fluid 36 through the processing space 12.

Referring again to FIGS. 6 and 7, the sanitizing phase 98 of the wash cycle 120 typically operates after a fill of wash fluid 36 within the processing space 12. In this manner, the wash fluid 36 can be filled to a level that at least partially submerges the sanitizing mechanism 10 under a surface of the wash fluid 36. As described herein, by submerging the sanitizing mechanism 10 below the surface of the wash fluid 36, the waves 48 of the ultrasonic frequency can be directed through the wash fluid 36 for producing the microbubbles 52 and also the cavitation 54 of the microbubbles 52 that produces the micro jets 56 of air 58.

Additionally, as exemplified in FIG. 6, the sanitizing phase 98 typically occurs after an agitation phase 150 and before a subsequent rinse phase 152. It is also contemplated that the rinse phase 152 may occur before the sanitizing phase 98 is initiated so that larger particles 110 that may be dislodged from the articles 18 can be drained from the processing space 12. Additionally, at the beginning of the sanitizing phase 98, new or filtered wash fluid 36 can be added to the processing space 12. During this sanitizing phase 98, as exemplified in FIGS. 6 and 7, the agitator 40 operates intermittently and in a pattern of oscillations and rotational movements. These rotational movements can be in opposing rotational directions to produce the rearranging effect with respect to the articles 18 being processed. Accordingly, over the course of the sanitizing phase 98, most, if not all of the various portions of the articles 18 being processed, can be acted upon by the micro jets 56 of air 58 that are produced by the cavitation 54 of microbubbles 52, as described herein. Using the operation of the rotator 38, and where utilized, the rotation of the drum 30, the treated portions 100 of the articles 18 are placed in a close proximity to the sanitizing mechanism 10. Accordingly, the waves 48 of the ultrasonic frequency can act upon the wash fluid 36 within this vicinity, which in turn acts upon the articles 18 being processed in the form of the micro jets 56 of air 58.

Referring again to FIGS. 1-7, during the sanitizing phase 98 of the laundry appliance 14, the ultrasonic transducers 46 of the sanitizing mechanism 10 are typically activated throughout the entire sanitizing phase 98. Through this configuration, as the articles 18 are rearranged in the processing space 12, the treated portions 100 of the articles 18 are continually being acted upon by the micro jets 56 of air 58 produced as a result of the waves 48 of the ultrasonic frequency from the ultrasonic transducers 46.

Referring again to FIGS. 6 and 7, where the sanitizing phase 98 is part of a comprehensive wash cycle 120, the sanitizing phase 98 typically occurs after the agitation phase 150 where larger particulate can be removed from the articles 18 being processed. As described herein, this agitating phase can be followed by a draining of wash fluid 36 from the processing space 12. In certain aspects of the device, the articles 18 are left saturated with wash fluid 36. In such an aspect of the device, the waves 48 of ultrasonic frequency produced by the sanitizing mechanism 10 can act on the wash fluid 36 that is held within the fabric of the articles 18 to produce the micro jets 56 of air 58. It is also contemplated that the agitating phase can be followed by a draining of the used wash fluid 36 and a refilling of the processing space 12 with new or filtered wash fluid 36. After the conclusion of the sanitizing phase 98, a rinse phase 152 is typically operated to remove soils and particulate of varying sizes from the processing space 12. This rinse that follows the sanitizing phase 98 is typically a rinse and spin phase that extracts a vast majority of the wash fluid 36, and captured particles 110, from the processing space 12 as well as the articles 18 being processed within the drum 30.

As exemplified in FIG. 7, where the sanitizing phase 98 is a dedicated cycle 122, the processing space 12 is first filled with wash fluid 36 to a level that submerges the sanitizing mechanism 10 below the wash fluid 36. At the conclusion of the sanitizing phase 98, the wash fluid 36 is drained from the processing space 12. Where the articles 18 being processed are in the form of clothing and other soft-type articles, this draining of wash fluid 36 can be in the form of a rinse and spin cycle that extracts a vast majority of the wash fluid 36 from the articles 18. In certain aspects of the device, the articles 18 being processed can be in the form of jewelry, household items, and other hard surfaces or potentially fragile items. In these instances, the draining of wash fluid 36 is accomplished only by operating a valve and a pump that directs the wash fluid 36 away from the processing space 12.

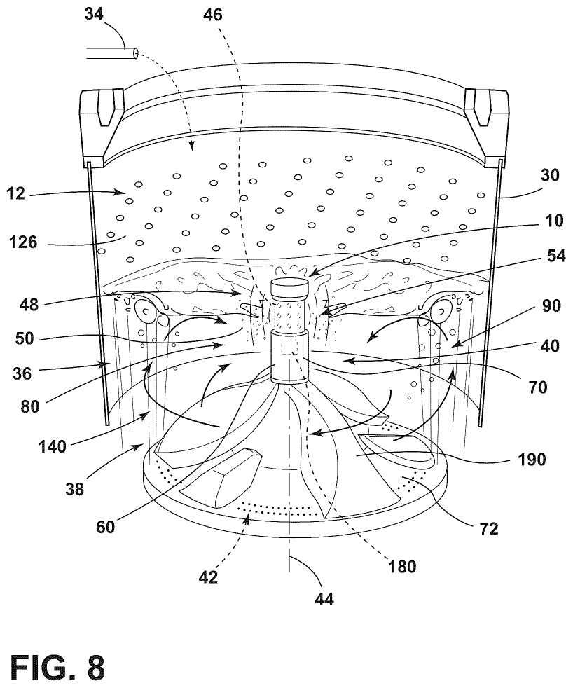

Referring now to FIGS. 8-10, it is contemplated that the sanitizing mechanism 10 can be included within various portions of a laundry appliance 14 for producing the waves 48 of the ultrasonic frequency. As exemplified in FIG. 8, the sanitizing mechanism 10 can be included within the post 70 of the agitator 40. In this configuration of the device, the waves 48 of the ultrasonic frequency are directed from the post 70 and are directed to radiate outward in the three-dimensional configuration 80 described herein. This configuration of the sanitizing mechanism 10 is able to locate the ultrasonic transducers 46 within the lower portion 90 of the processing space 12 for sanitizing smaller loads of articles 18 within the processing space 12. In certain aspects of the device, the ultrasonic transducers 46 can be situated in various locations within the post 70 of the agitator 40. By way of example, and not limitation, the post 70 of the agitator 40 may include a series of receivers 60 that allow for placement of the ultrasonic transducers 46 at various heights with respect to the processing space 12. It is also contemplated that ultrasonic transducers 46 can be located within various locations of the post 70 such that the waves 48 of the ultrasonic frequency can emanate from any one of various locations along the post 70 of the agitator 40.

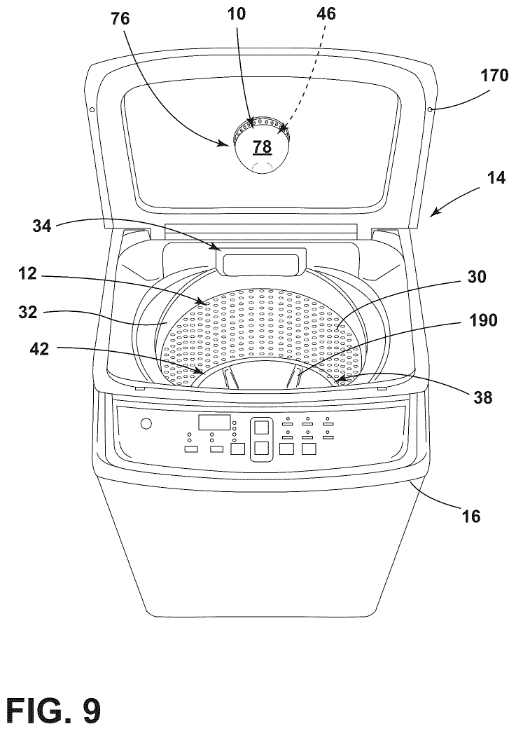

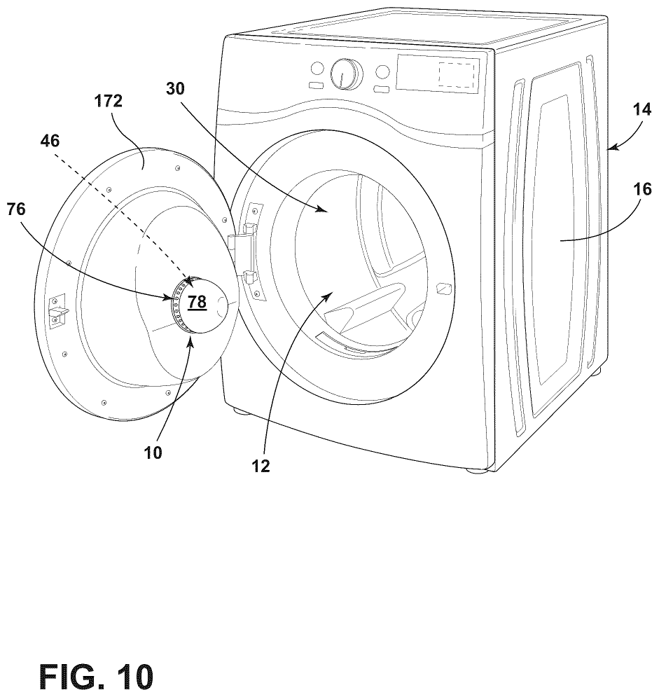

In certain aspects of the device, the sanitizing mechanism 10 having the ultrasonic transducers 46 can also be located on a portion of the appliance 14 other than, or in addition to, the rotator 38. As exemplified in FIGS. 9 and 10, the sanitizing mechanism 10 can be located on a lid 170 or door 172 of the rotating appliance 14 such that as wash fluid 36 engages these portions of the appliance 14, the waves 48 of the ultrasonic frequency can be directed through the wash fluid 36 for acting on certain portions of the articles 18 being processed.

As exemplified in FIG. 9, the sanitizing mechanism 10 can be attached to a lid 170 or a vertical-axis appliance 14 where the lid 170 is disposed above the processing space 12. In such an aspect of the device, the sanitizing mechanism 10 can be attached to the lid 170. It is also contemplated that the sanitizing mechanism 10 can be configured to extend downward from the lid 170 to be placed within the wash fluid 36 within the processing space 12. In such a configuration, the sanitizing mechanism 10 can be attached and detached from the lid 170 as needed for operating the various sanitization operations for the laundry appliance 14.

Referring again to FIGS. 1-10, the sanitizing mechanism 10 for the appliance 14 is configured to be selectively removable from the rotator 38 or other portion of the appliance 14. In this manner, the sanitizing mechanism 10 requires electrical power for operating the ultrasonic transducers 46. This electrical power can be provided to the ultrasonic transducers 46 through a self-contained power source, such as a battery 180, within the sanitizing mechanism 10. It is also contemplated that the electrical power for operating the ultrasonic transducers 46 can be obtained from the appliance 14.

By way of example, and not limitation, the sanitizing mechanism 10 that can be attached to the receiver 60 within the rotator 38 can be a battery-powered module that includes the self-contained power source for operating the ultrasonic transducers 46. It is also contemplated that the sanitizing mechanism 10 can be a rechargeable module that can be installed within a recharging station external to the appliance 14. When not in use, the sanitizing module can be placed within the recharging station for charging an internal battery 180 that operates and powers the ultrasonic transducers 46. In other aspects of the device, the various receiver 60 or receivers 60 included within the rotator 38 can include electrical contacts that can be used to attach to the sanitizing mechanism 10. These contacts can be used to deliver electrical power, as well as one-way, or two-way communications between the sanitizing mechanism 10 and the appliance 14.

Referring again to FIGS. 1-10, the ultrasonic transducer 46 can be attached to a receiver 60 that is incorporated within an agitator 40 for the laundry appliance 14. This agitator 40 includes a lower section, typically in the form of an impeller-type section that includes raised fins 190. As described herein, these raised fins 190 assist in producing the toroidal flow 140 of wash fluid 36 that helps in rearranging and redistributing the articles 18 being processed within the processing space 12. The post 70 extends upward from the lower section of the agitator 40. An ultrasonic transducer assembly, which can be in the form of the sanitizing mechanism 10, is attached to the post 70. The ultrasonic transducer assembly 10 is positioned to direct the waves 48 of the ultrasonic frequency in the three-dimensional configuration 80 that radiates outward from the post 70 and a wide range and combination of vertical and lateral directions 84, 82. The ultrasonic transducer assembly 10 is selectively removable from the receiver 60 defined within the post 70 for the agitator 40. The ultrasonic transducer assembly 10 also delivers the waves 48 of the ultrasonic frequency into an amount of wash fluid 36 that is disposed within the processing space 12.

As described herein, this amount of wash fluid 36 is typically sufficient to submerge the ultrasonic transducer assembly 10 within the wash fluid 36. The waves 48 of the ultrasonic frequency are configured to generate air bubbles 50 within the wash fluid 36. Additionally, the waves 48 of the ultrasonic frequency also generate a cavitation effect that causes the air bubbles 50 to collapse. This collapse or cavitation 54 of the air bubbles 50 directs a micro jet 56 of air 58 through the wash fluid 36 and into articles 18 being processed within the processing space 12.

As described herein, the ultrasonic transducer assembly 10 typically includes a generally spheroid configuration that radiates the waves 48 of the ultrasonic frequency in the three-dimensional configuration 80. This three-dimensional configuration 80 of the waves 48 can be in a generally spherical configuration, a semi-spherical configuration, or other three-dimensional configurations 80 that direct the waves 48 in a plurality of directions around the ultrasonic transducer assembly 10.

As described herein, and as exemplified in FIGS. 1-10, the sanitizing mechanism 10 can be disposed within any one of various appliances that are used to clean and/or sanitize articles 18 within a particular processing space 12 within an outer cabinet 16. These appliances 14 can be generally characterized as cleaning appliances. These cleaning appliances can include a tub 32 that is disposed within a structural cabinet, where the tub 32 defines at least a portion of the processing space 12. The fluid delivery system 34 selectively delivers wash fluid 36 to the processing space 12. It is contemplated that the wash fluid 36 described herein can be in the form of water, cleaning chemistry, accumulated soils, and other similar materials that can be used to form the various configurations and components of the wash fluid 36. Accordingly, this wash fluid 36 can be obtained from a fluid source, such as a well or municipal water supply, or can be recirculated through the appliance 14. The various cleaning chemistries can be in the form of laundry chemistry, detergent, bleach, fabric softener, liquid or powered cleaning agents, combinations thereof, and other similar cleaning chemistries.

Referring again to FIGS. 1-10, the cleaning appliance can include the rotator 38 that rotationally operates within the processing space 12. This rotator 38 selectively operates within the processing space 12 and about the rotational axis 44. Through this operation of the rotator 38, the articles 18 being processed can be manipulated, agitated, and otherwise rearranged within the processing space 12. By being rearranged, various portions of the articles 18 are moved throughout the processing space 12 to come into close proximity with the sanitizing mechanism 10 at least at some point or a plurality of points throughout the sanitizing phase 98 of the appliance 14. The sanitizing mechanism 10 that is included within the processing space 12 includes an ultrasonic transducer 46 or a plurality of ultrasonic transducers 46. The ultrasonic transducer 46 selectively delivers the waves 48 of the ultrasonic frequency into an amount of the wash fluid 36 that is disposed within the processing space 12. The waves 48 of the ultrasonic frequency operate on the wash fluid 36 to generate air bubbles 50, typically in the form of microbubbles 52 within the wash fluid 36. The waves 48 of the ultrasonic frequency also act on these microbubbles 52 to cause a cavitation action with respect to the air bubbles 50. This cavitation 54 causes the air bubbles 50 to collapse and, in turn, produce a micro jet 56 of air 58 that is directed through the wash fluid 36 and typically toward a portion of the articles 18 being processed. As described herein, the sanitizing mechanism 10 is selectively removable from a receiver 60 that is defined within the rotator 38.

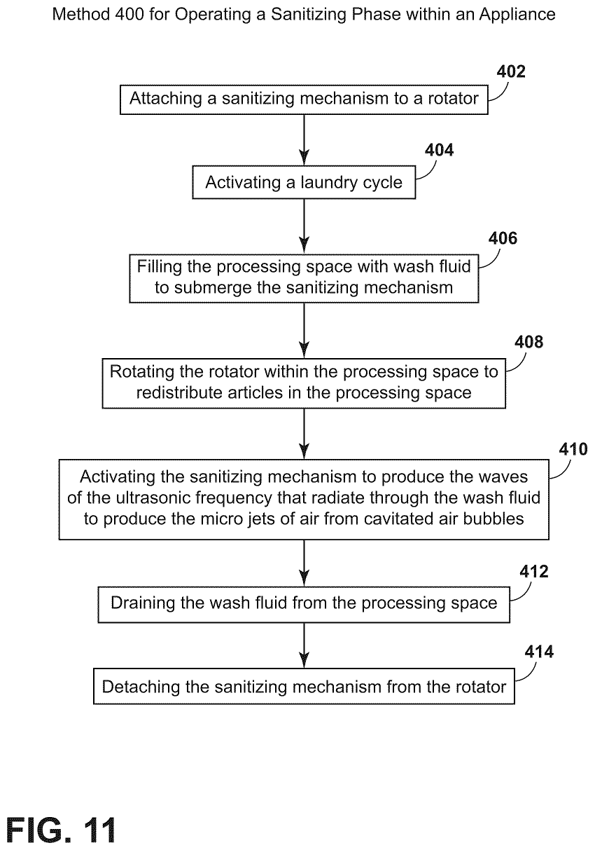

Referring now to FIGS. 1-11, having described various aspects of the sanitizing mechanism 10 and operating the sanitizing phase 98 within an appliance 14, a method 400 is described for operating a sanitizing phase 98 of the appliance 14. According to the method 400, step 402 includes attaching the sanitizing mechanism 10 to the rotator 38. As described herein, the sanitizing mechanism 10 is attached to a receiver 60 of the rotator 38 and can be selectively attached and removed from the rotator 38 as needed for operating the sanitizing phase 98. Once the sanitizing mechanism 10 is attached to the rotator 38, a cycle of the laundry appliance 14 can be activated (step 404). This cycle can be in the form of a comprehensive wash cycle 120, where a sanitizing phase 98 is incorporated as an aspect of this wash cycle 120. Alternatively, the cycle can be in the form of a dedicated cycle 122 that includes the sanitizing phase 98. According to the method 400, when the wash cycle 120 (or the dedicated cycle 122) is activated, the processing space 12 is filled with wash fluid 36 to a level that submerges or at least partially submerges the sanitizing mechanism 10 within the wash fluid 36 (step 406). As described herein, this allows the waves 48 of the ultrasonic frequency to be rated through the wash phase to produce the microbubbles 52 that can undergo the cavitation 54 to produce the micro jets 56 of air 58. Step 408 includes rotating the rotator 38 within the processing space 12 to rearrange and redistribute the articles 18 being processed. Step 410 includes activating the sanitizing mechanism 10 to produce the waves 48 of the ultrasonic frequency that radiate through the wash fluid 36. According to the method 400, when the sanitizing phase 98 is near completion, the wash fluid 36 is drained from the processing space 12 (step 412). As the wash fluid 36 is drained, captured particulate that has been removed from the article 18 that is being processed is also drained away from the articles 18 and away from the processing space 12. At the conclusion of the sanitizing phase 98, or the wash cycle 120, the sanitizing mechanism 10 is detached from the rotator 38 (step 414). In this manner, the sanitizing mechanism 10 can be positioned somewhere external to the appliance 14, such as in a recharging station or storage area when not in use.

As described herein, the articles 18 being processed can be in the form of clothing-type articles, as well as non-clothing articles such as jewelry, household items, dishes, glasses, watches, collectibles, combinations thereof, and other similar items that may be cleaned and sanitized within the appliance 14.

According to various aspects of the device, it is contemplated that the wash fluid 36 used during a sanitizing phase 98 can be heated to a predetermined temperature. By using this heated water, the ultrasonic transducers 46 and the waves 48 of the ultrasonic frequency can have a more effective performance at producing the microbubbles 52 and also the cavitation 54 effect that produces the micro jets 56 of air 58. Additionally, the use of heated wash fluid 36 within the processing space 12 and during the sanitizing phase 98 can at least partially kill, immobilize, or otherwise mitigate the effect of bacteria, viruses, and other microbes that may be present within the processing space 12.

According to various aspects of the device, the ultrasonic transducers 46 can operate at any one of various frequencies for producing the waves 48 of the ultrasonic frequency. Such frequencies can include, those frequencies above approximately 15 KHz. This sanitizing phase 98 can also operate for a time period of approximately three (3) minutes to twenty (20) minutes, or other similar time periods. Additionally, as described herein, the wash fluid 36 can include various cleaning chemistries. In certain aspects, the cleaning chemistries can be added as a component of the wash fluid 36 to be disposed within the processing space 12 during the sanitizing phase 98. These cleaning chemistries, such as detergent, can produce additional amount of microbubbles 52 that can be acted upon by the waves 48 of the ultrasonic frequency. By adding more microbubbles 52 to the wash fluid 36, the number of micro jets 56 of air 58 produced by the cavitation 54 of these microbubbles 52 can be increased.

Utilizing the sanitizing mechanism 10 and the ultrasonic transducers 46 for producing the waves 48 of the ultrasonic frequency, articles 18 that are processed within the processing space 12 can be cleaned and sanitized with a minimal amount of agitation applied to the various articles 18. Accordingly, utilizing the sanitizing mechanism 10, less wear and tear are placed upon the articles 18 during a wash cycle 120. At the same time, greater efficiency and effectiveness of cleaning and sanitizing are achieved without increasing the length and/or intensity of an agitating function 124 of a wash cycle 120. Also, laundry items can be cleaned and sanitized more effectively and over a shorter prior of time such that less water is used and the water that is used as a component of the wash fluid 36 is used more effectively and efficiently throughout the course of the wash cycle 120 and through dedicated sanitizing phases 98.

The invention disclosed herein is further summarized in the following paragraphs and is further characterized by combinations of any and all of the various aspects described therein.

According to one aspect of the present disclosure, a laundry appliance includes a drum that rotationally operates within a tub. A fluid delivery system selectively delivers wash fluid to the drum. A rotator rotationally operates within the drum. The rotator selectively operates independent of the drum and about a common rotational axis of the drum and the rotator. A sanitizing mechanism includes an ultrasonic transducer. The ultrasonic transducer selectively delivers waves of an ultrasonic frequency into an amount of the wash fluid disposed within the drum and the rotator. The ultrasonic frequency generates air bubbles and causes cavitation of the air bubbles that directs a micro jet of air through the wash fluid and into articles being processed within the drum.

According to another aspect, the rotator is an agitator. The sanitizing mechanism is selectively removable from the rotator.

According to another aspect, the sanitizing mechanism is incorporated within a cylindrical portion of the agitator.

According to another aspect, the sanitizing mechanism is disposed at a top portion of the agitator.

According to another aspect, the sanitizing mechanism is a generally spheroid member.

According to another aspect, the ultrasonic transducer is positioned within the drum to at least partially submerge the ultrasonic transducer within the wash fluid during a sanitizing portion of a wash cycle.

According to another aspect, the ultrasonic transducer is activated during the sanitizing portion of the wash cycle. The sanitizing portion is characterized by a rotational operation of at least one of the drum and the rotator.

According to another aspect, the wash fluid used during the sanitizing portion of the wash cycle is heated wash fluid.

According to another aspect, the sanitizing portion of the wash cycle occurs after an agitating phase of the wash cycle and before a rinse phase of the wash cycle.

According to another aspect of the present disclosure, an agitator for a laundry appliance includes a lower section that includes raised fins, a post that extends upward from the lower section, and an ultrasonic transducer assembly that is attached to the post. The ultrasonic transducer assembly is positioned to direct waves of an ultrasonic frequency in a three-dimensional configuration that radiates from the post. The ultrasonic transducer assembly is selectively removable from a receiver that is defined within the post. The ultrasonic transducer assembly delivers the waves of the ultrasonic frequency into an amount of wash fluid that is disposed within a processing space. The waves of the ultrasonic frequency are configured to generate air bubbles and cause cavitation of the air bubbles that directs a micro jet of air through the wash fluid and into articles that are processed within the processing space.

According to another aspect, the ultrasonic transducer assembly includes a generally spheroid configuration that radiates the waves of the ultrasonic frequency in the three-dimensional configuration. The three-dimensional configuration is generally semi-spherical.

According to another aspect, the ultrasonic transducer assembly is positioned at a top of the post.

According to another aspect, the ultrasonic transducer assembly is positioned within the post.

According to another aspect, the ultrasonic transducer assembly is configured to be at least partially submerged in the wash fluid for directing the waves of the ultrasonic frequency through the wash fluid.

According to yet another aspect of the present disclosure, a cleaning appliance includes a tub disposed within a structural cabinet. The tub defines a processing space. A fluid delivery system selectively delivers wash fluid to the processing space. A rotator rotationally operates within the processing space. The rotator selectively rotates within the processing space about a rotational axis. A sanitizing mechanism includes an ultrasonic transducer. The ultrasonic transducer selectively delivers waves of an ultrasonic frequency into an amount of the wash fluid disposed within the processing space. The waves of the ultrasonic frequency generate air bubbles and cause cavitation of the air bubbles that directs a micro jet of air through the wash fluid and into articles that are processed within the processing space. The sanitizing mechanism is selectively removable from a receiver that is defined within the rotator.

According to another aspect, the articles being processed include non-clothing articles that are selectively disposed within the processing space.

According to another aspect, the rotator is an agitator that is disposed within a rotating drum that defines at least a portion of the processing space.

According to another aspect, the sanitizing mechanism is disposed at a top portion of the agitator.

According to another aspect, the ultrasonic transducer is positioned within the processing space and is configured to be selectively submerged within the wash fluid during a sanitizing cycle.

According to another aspect, the wash fluid used during a sanitizing portion of the wash cycle is heated wash fluid.

It will be understood by one having ordinary skill in the art that construction of the described disclosure and other components is not limited to any specific material. Other exemplary embodiments of the disclosure disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure as shown in the exemplary embodiments is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

What is claimed is:

1. A laundry appliance comprising:
a drum that rotationally operates within a tub;
a fluid delivery system that selectively delivers wash fluid to the drum;
a rotator that rotationally operates within the drum, wherein the rotator selectively operates independent of the drum and about a common rotational axis of the drum and the rotator; and
a sanitizing mechanism that includes an ultrasonic transducer, wherein the ultrasonic transducer selectively delivers waves of an ultrasonic frequency into an amount of the wash fluid disposed within the drum and the rotator, the ultrasonic frequency generating air bubbles and causing cavitation of the air bubbles that directs a micro jet of air through the wash fluid and into articles being processed within the drum.

2. The laundry appliance of claim 1, wherein the rotator is an agitator, wherein the sanitizing mechanism is selectively removable from the rotator.

3. The laundry appliance of claim 2, wherein the sanitizing mechanism is incorporated within a cylindrical portion of the agitator.

4. The laundry appliance of claim 2, wherein the sanitizing mechanism is disposed at a top portion of the agitator.

5. The laundry appliance of claim 2, wherein the sanitizing mechanism is a generally spheroid member.

6. The laundry appliance of claim 2, wherein the ultrasonic transducer is positioned within the drum to at least partially submerge the ultrasonic transducer within the wash fluid during a sanitizing phase of a wash cycle.

7. The laundry appliance of claim 6, wherein the ultrasonic transducer is activated during the sanitizing phase of the wash cycle, wherein the sanitizing phase is characterized by a rotational operation of at least one of the drum and the rotator.

8. The laundry appliance of claim 6, wherein the wash fluid used during the sanitizing phase of the wash cycle is heated wash fluid.

9. The laundry appliance of claim 6, wherein the sanitizing phase of the wash cycle occurs after an agitating phase of the wash cycle and before a rinse phase of the wash cycle.

10. An agitator for a laundry appliance, the agitator comprising:
a lower section that includes raised fins;
a post extending upward from the lower section; and
an ultrasonic transducer assembly, attached to the post, wherein the ultrasonic transducer assembly is positioned to direct waves of an ultrasonic frequency in a three-dimensional configuration that radiates from the post, wherein
the ultrasonic transducer assembly is selectively removable from a receiver defined within the post;
the ultrasonic transducer assembly delivers the waves of the ultrasonic frequency into an amount of wash fluid disposed within a processing space; and
the waves of the ultrasonic frequency are configured to generate air bubbles and cause cavitation of the air bubbles that directs a micro jet of air through the wash fluid and into articles being processed within the processing space.

11. The agitator of claim 10, wherein the ultrasonic transducer assembly includes a generally spheroid configuration that radiates the waves of the ultrasonic frequency in the three-dimensional configuration, and wherein the three-dimensional configuration is generally semi-spherical.

12. The agitator of claim 11, wherein the ultrasonic transducer assembly is positioned at a top of the post.

13. The agitator of claim 10, wherein the ultrasonic transducer assembly is positioned within the post.

14. The agitator of claim 10, wherein the ultrasonic transducer assembly is configured to be at least partially submerged in the wash fluid for directing the waves of the ultrasonic frequency through the wash fluid.

15. A cleaning appliance comprising:
a tub disposed within a structural cabinet, wherein the tub defines a processing space;
a fluid delivery system that selectively delivers wash fluid to the processing space;
a rotator that rotationally operates within the processing space, wherein the rotator selectively rotates within the processing space about a rotational axis; and
a sanitizing mechanism that includes an ultrasonic transducer, wherein the ultrasonic transducer selectively delivers waves of an ultrasonic frequency into an amount of the wash fluid disposed within the processing space, the waves of the ultrasonic frequency generating air bubbles and causing cavitation of the air bubbles that directs a micro jet of air through the wash fluid and into articles being processed within the processing space, wherein the sanitizing mechanism is selectively removable from a receiver defined within the rotator.

16. The cleaning appliance of claim 15, wherein the articles being processed includes non-clothing articles that are selectively disposed within the processing space.

17. The cleaning appliance of claim 15, wherein the rotator is an agitator that is disposed within a rotating drum that defines at least a portion of the processing space.

18. The cleaning appliance of claim 17, wherein the sanitizing mechanism is disposed at a top portion of the agitator.

19. The cleaning appliance of claim 15, wherein the ultrasonic transducer is positioned within the processing space and is configured to be selectively submerged within the wash fluid during a sanitizing phase of the wash cycle.

20. The cleaning appliance of claim 19, wherein the wash fluid used during the sanitizing phase of the wash cycle is heated wash fluid.
